(19) United States
(12) Patent Application Publication
(10) Pub. No.: US 2002/0028496 A1
(43) Pub. Date: Mar. 7, 2002

(54) Method for producing telomerase reverse transcriptase

(76) Inventors: Seishi Murakami, Ishikawa (JP); Shuichi Kaneko, Ishikawa (JP); Kenkichi Masutomi, Ishikawa (JP)

Correspondence Address: SUGHRUE, MION ZINN, MACPEAK & SEAS, PLLC, 2100 Pennsylvania Avenue, N.W., Washington, DC, 20037-3213, US

(21) Appl. No.: 09/968,506

(22) Filed: Oct. 2, 2001

Related U.S. Application Data
(63) Continuation-in-part of application No. PCT/JP00/00554, filed on Feb. 2, 2000.

(51) Int. Cl. C12N 9/22; C12P 21/02; C12N 15/866

(52) U.S. Cl. 435/199; 435/69.1; 435/325; 435/456; 435/320.1

(57) ABSTRACT

A method for producing telomerase reverse transcriptase comprising the steps of infecting an insect cell with a baculovirus containing a DNA encoding telomerase reverse transcriptase, and then culturing the infected cell to express the telomerase reverse transcriptase is disclosed. Further, a method for solubilizing telomerase reverse transcriptase comprising treating an insect cell expressing telomerase reverse transcriptase or a lysate thereof with an aqueous solution containing N-D-gluco-N-methylalkanamide of the formula (I):

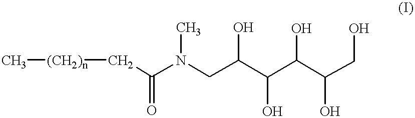

wherein n denotes an integer of 5 to 7 is also disclosed.

### BACKGROUND OF THE INVENTION

1. Field of the Invention

The present invention relates to a novel method for producing telomerase reverse transcriptase, particularly to a novel method for expressing or solubilizing telomerase revere transcriptase.

2. Description of the Related Art

Telomere has a specialized structure at the ends of linear eukaryotic chromosomes. It provides a mechanism for maintaining chromosome length, and has critical functions in maintaining chromosome stability. Telomere structure is composed of long tandem repeats (TTAGGG), telomere repeats, and specific DNA-binding proteins. Elongation of telomere repeat regions is distinct from the semi-conserved chromosome replication, and is carried out by a catalytic reaction with telomerase, RNA dependent DNA synthesis, that is, a reverse transcription. A ribonucleoprotein complex, telomerase, elongates telomere and is composed of a template RNA (hereinafter referred to as “TR”, and human TR to as “hTR”) and several proteins. Telomerase reverse transcriptase (hereinafter referred to as “TERT” and human TERT to as “hTERT”) has been identified as a catalytic enzyme of telomere elongation. TERT contains motifs found in many reverse transcriptases, and these motifs are highly conserved among those from the budding yeast to human.

Human TERT is a rate-limiting factor for a telomerase activity both biologically and enzymatically. Introduction of hTERT into normal human primary cells overcomes. senescence and extends their lifespan, and that of hTERT into transformed cells caused cellular immortalization without crisis. Transient expression of hTERT in telomerase-negative normal human cells generates a telomerase activity therein. Telomerase is highly active in most cancer cells and immortalized cells, whereas a telomerase activity is suppressed in somatic cells. Thus, hTERT may play an important role for cellular senescence and carcinogenesis.

Recently, some groups reported an in vitro reconstitution of telomerase, and demonstrated the essential roles of hTERT and human telomerase RNA, hTR, as an RNA template. One group demonstrated that Hsp90 and p23 were essential in addition to hTR for a telomerase activity, using a recombinant hTERT synthesized de novo in rabbit reticulocyte lysates in vitro. The other group reported that the production of an active recombinant telomerase of Tetrahymena required a factor in rabbit reticulocyte lysate that promoted ribonucleoprotein assembly. In the latter two systems, certain factor(s) carried over with rabbit reticulocytes may influence the native telomerase activity. Therefore, it remains obscure that these two components, hTERT and hTR, are sufficient for in vitro telomerase reconstitution. Purified hTERT and hTR are necessary to clarify the above problem and, furthermore, provide an experimental system to identify factors which are essential or stimulatory for the telomerase activity in vitro.

### SUMMARY OF THE INVENTION

Under the circumstances, the inventors of the present invention had tried several bacterial expression and purification procedures, but been unsuccessful.

Thus, the present inventors engaged in intensive research to develop preparation of purified hTERT, and as a result, found that a large amount of hTERT can be expressed in insect cells, using the baculovirus expression system. Further, the present inventors found the optimum conditions of infection and cell culture which are quite different from the usual ones.

Importantly, the present inventors discovered that the target protein (hTERT) expressed in the insect cells as mentioned above cannot be solubilized with non-ionic detergents (such as Triton X-100, Nonidet P-40, or CHAPS) generally employed in usual purification of proteins, but can be solubilized with specific non-ionic detergents.

The present invention is based upon these findings.

Accordingly, the object of the present invention is to provide a method for producing TERT in a large scale.

The another object of the present invention is to provide a method for solubilizing the TERT obtained under an unsolubilized state according to the above method.

Other objects and advantages of the present invention will be apparent from the following description.

In accordance with the present invention, there is provided a method for producing telomerase reverse transcriptase comprising the steps of: infecting an insect cell with a baculovirus containing a DNA encoding telomerase reverse transcriptase, and then culturing the infected cell to express the telomerase reverse transcriptase.

Further, the present invention relates to a method for solubilizing telomerase reverse transcriptase comprising treating an insect cell expressing telomerase reverse transcriptase or a lysate thereof with an aqueous solution containing N-D-gluco-N-methylalkanamide of the formula (I):

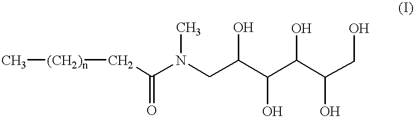

wherein n denotes an integer of 5 to 7.

Further, the present invention also relates to a method for producing telomerase reverse transcriptase comprising the steps of:

infecting an insect cell with a baculovirus containing a DNA encoding telomerase reverse transcriptase,

culturing the infected cell to express the telomerase reverse transcriptase, and then,

treating the insect cell expressing telomerase reverse transcriptase or a lysate thereof with an aqueous solution containing N-D-gluco-N-methylalkanamide of the formula (I):

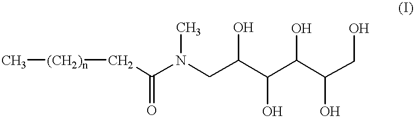

wherein n denotes an integer of 5 to 7.

Further, the present invention also relates to a baculovirus expression vector containing a DNA encoding telomerase reverse transcriptase. Further, the present invention also relates to a baculovirus containing a DNA encoding telomerase reverse transcriptase. Further, the present invention also relates to an insect cell containing the baculovirus as above.

### BRIEF DESCRIPTION OF DRAWINGS

FIG. 1 is a photograph showing the whole lysates of High5 insect cells infected with the recombinant baculovirus BVKM-FLAG-hTERT in varying multiplicity of infection, which were fractionated by gel electrophoresis.

FIG. 2 is a photograph showing the whole lysates of High5 insect cells infected with the recombinant baculovirus BVKM-FLAG-hTERT in varying incubation time, which were fractionated by gel electrophoresis.

FIG. 3 is a photograph showing the fractions in the purification steps, which were fractionated by gel electrophoresis.

FIG. 4 is a photograph showing the whole lysate of infected insect cells, and the partially purified preparation of the FLAG-hTERT fusion protein, which were fractionated by gel electrophoresis.

FIG. 5 is a photograph showing in vitro reconstituted telomerase activity detected by a TRAP assay, of which products were fractionated by gel electrophoresis.

FIG. 6 is a photograph showing time course of an in vitro reconstituted telomerase activity detected by a TRAP assay, of which products were fractionated by gel electrophoresis.

FIG. 7 is a graphical presentation showing in vitro reconstituted telomerase activity detected by a TRAP ELISA method, of which products were fractionated by gel electrophoresis.

FIG. 8 is a photograph showing the purified wild and mutant (D712) FLAG-hTERT fusion proteins that were fractionated by gel electrophoresis.

FIG. 9 is a photograph in place of a drawing showing telomerase activities of TIG3 cell extract and the purified wild or mutant (D712) FLAG-hTERT fusion protein detected by a TRAP assay, of which products were fractionated by gel electrophoresis.

FIG. 10 is a photograph showing in vitro reconstituted telomerase activities supplemented by TIG3 cell extract detected by a TRAP assay, of which products were fractionated by gel electrophoresis.

FIG. 11 is a graphical presentation showing in vitro reconstituted telomerase activities supplemented by TIG3 cell extract detected by a TRAP ELISA, of which products were fractionated by gel electrophoresis.

FIG. 12 is a photograph showing a telomerase activity in the presence of Geldanamycin (GA) detected by a TRAP assay, of which products were fractionated by gel electrophoresis.

FIG. 13 is a graphical presentation showing a telomerase activity in the presence of Geldanamycin (GA) detected by TRAP ELISA, of which products were fractionated by gel electrophoresis.

FIG. 14 is a photograph showing the labeled RNA probes fractionated by gel electrophoresis, which were used for Electrophoretic Motility Shift Assay (hereinafter referred to as EMSA).

FIG. 15 is a photograph showing the complex formation of the FLAG-hTERT fusion protein and hTR in vitro detected by EMSA.

### DESCRIPTION OF THE PREFERRED EMBODIMENTS

The production method of telomerase reverse transcriptase of the present invention includes synthesis procedure of telomere reverse transcriptase by expressing telomerase reverse transcriptase using insect cells (hereinafter referred to as “the synthetic procedure”), and solubilization procedure of telomerase reverse transcriptase expressed in insect cells as mentioned above (hereinafter referred to as “the solubilization procedure”). The synthetic procedure and the solubilization procedure are by themselves a novel synthetic method and a novel solubilization method.

The term “telomerase reverse transcriptase” as used herein includes natural telomerase reverse transcriptases (native proteins having a reverse transcriptase activity to elongate telomere in the presence of TR) of biological species (including animals, plants, and microorganisms), and also includes functionally equivalent modified proteins thereof, for example, proteins derived from natural telomerase reverse transcriptases by substitution, deletion and/or addition of one or more (preferably one or several) amino acids in the amino acid sequence of the natural telomerase reverse transcriptases, and having the telomerase activity, a part or a fragment of the natural telomerase reverse transcriptase or the functionally equivalent modified proteins thereof, and fused forms of the natural or functionally equivalent modified proteins, for instance, fused proteins with tagging proteins, such as FLAG-peptide or glutation-S-transferase.

Baculoviruses which may be used in the synthetic procedure of the present invention are not particularly limited, so long as they may be used for usual baculovirus expression systems, such as a nuclear polyhedrosis virus (hereinafter referred to as NPV) of Bombyx mori (BMNPV) or an NPV of Autographa californica (AcNPV).

As baculovirus expression vectors (i.e., transfer vectors) to construct recombinant baculovirus having the cDNA encoding telomerase reverse transcriptase, the baculovirus-derived transfer vectors listed above, for example, PVL1392 and PVL1393, may be used.

Host cells which may be used in the synthetic procedure of the present invention are not particularly limited, so long as they are insect cells which may be used for usual baculovirus expression systems such as cells derived from Trichoplusia ni, those from Spodoptera grugiperda, or those from Bombyx mori.

For more efficient expression of the target protein, the tissue cultures from Trichoplusia ni are preferable, and those from oocytes of the same species are more preferable, and High5 strain which was derived from oocytes of the same species and was treated by the methods mentioned in the reports, Biotechnol. Prog., 8, 391-396, 1992, and In vitro Cells & Dev., 29A, 388-390, 1993, is most preferable. High5 cell strain is commercially available from, for example, Invitrogen (Code number:BTI-TN-5BI-4).

After inserting the DNA sequence encoding telomere reverse transcriptase into the transfer vectors as above mentioned, and constructing vectors having the cDNA sequence encoding telomere reverse transcriptase, baculovirus having the DNA sequence encoding telomere reverse transcriptase can be prepared by homologous recombination with the usual method.

In the synthetic procedure of the present invention, multiplicity of infection (moi: hereinafter referred to as moi) is not particularly limited. For example, infection can be carried out at moi 0.1 to 10, preferably 0.1 and 0.3 or 5 to 10, more preferably 0.1 to 0.3. In the usual protocol, the optimum moi has been known to be between 5 and 10.

In the synthetic procedure of the present invention, incubation time after infection is not particularly limited. For example, it may be for 2 to 8 days, preferably for 4 to 6 days. In the usual protocol, the optimum incubation time has been known to be for 2 to 3 days.

In the above-mentioned synthetic procedure, telomerase reverse transcriptase expressed in the insect cells is accumulated as insoluble states in the cells. Therefore, it remains insoluble even by sonication of cells suspended in buffer, thus cannot be applied for the purification procedure.

In the solubilization procedure of the present invention, the above-mentioned insect cells expressing telomerase reverse transcriptase or the lysates thereof are solubilized by a solution containing N-D-gluco-N-methylalkanamide of the formula (I):

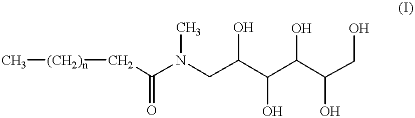

wherein n denotes an integer of 5 to 7.

N-D-Gluco-N-methylalkanamide of the above-mentioned formula (1) in the solubilization procedure includes n-octanoyl-N-methylglucamide (MEGA-8: the compound of the formula (1) when n is 5), n-nonanoyl-N-methylglucamide (MEGA-9: the compound of the formula (1) when n is 6), and n-decanoyl-N-methylgulamide (MEGA-10: the compound of the formula (1) when n is 7).

A concentration of N-D-glucomethylalkanamide of the above-mentioned formula (1) is not particularly limited so far as it is in a level that telomerase reverse transcriptase can be solubilized, for example, 0.5 to 2% (w/v), more preferably 0.5 to 1.0% (w/v).

The above-mentioned solubilization solution may contain one or more salts, proteinase inhibitors, reducing agents, and/or glycerol, if desired.

The above-mentioned salts may be a usual salt used for protein purifications, such as a halide of an alkaline metal or an alkaline earth metal.

The above-mentioned proteinase may be, for example, phenylmethanesulfonyl fluoride (PMSF), leupeptine, pepstatin, antipain, phenanthroline, or benzamide for example.

The above-mentioned reducing agent may be, for example, dithiothreitol (DTT) or 2-mercaptoethanol.

A concentration of the salt in the above-mentioned solubilization solution is not particularly limited, so far as it is in a level that telomerase reverse transcriptase can be solubilized, for example, 400 to 1200 mmol/L, preferably 500 to 1000 mmol/L.

A pH value of the above-mentioned solubilization solution is not particularly limited, so far as it is at a level that telomerase reverse transcriptase is not inactivated, but preferably pH around neutral, for example, pH 7.2 to 7.8.

A method for treating the target insect cells or lysates thereof with the above-mentioned solubilization solution in the solubilization procedure is not particularly limited, so far as it can solubilize telomerase reverse transcriptase in cells or lysates thereof.

When the target insect cells or cell lysates are in suspension, for example, solubilization can be carried out by mixing suspension with the solubilization solution. When the target samples for solubilization are in precipitate forms, for example, a precipitate fraction after centrifugation, then solubilization can be carried out by mixing the precipitates with the solubilization solution.

The solution obtained after the solubilization procedure in the present invention contains solubilized telomerase reverse transcriptase. The solution can be directly used as a fraction containing telomerase reverse transcriptase, or also can be subjected to further purify telomerase reverse transcriptase using available purification methods of protein (for example, fractionation with ammonium sulfate, chromatography, dialysis, and/or lyophilization).

The present inventors firstly discovered that telomerase reverse transcriptase can bind heparin or poly (U) (poly-uridylate) in a purification procedure thereof. Therefore, affinity chromatography using heparin Sepharose and/or poly (U)-Sepharose can be applied to purify telomerase reverse transcriptase in addition to the above-mentioned conventional methods. It is believed that the binding ability of hTERT to heparin is related to the fact that hTERT retains the motifs highly conserved among reverse transcriptases of various species from yeast to human, and the telomerase RNA component, i.e., hTR, is used as an RNA template in the telomerase reaction.

In the method of the present invention, the insect cells or lysates thereof obtained by the synthetic procedure can be directly subjected to the solubilization procedure, or can be subjected to partial purification processes at first, then fractionated products can be subjected to the solubilization procedure. By introducing appropriate partial purification steps before the solubilization procedure, some purification steps of telomerase reverse transcriptase after the solubilization procedure can be simplified or skipped.

For example, the solubilization procedure can be carried out by homogenizing the insect cells obtained in the synthetic procedure, centrifuging the homogenized product, removing the supernatant, and then using the resultant precipitate as the starting material of the solubilization procedure. In this case, most of soluble proteins contained in the homogenized product are removed with the supernatant, and therefore, procedures for removing soluble proteins can be omitted or simplified, when the telomerase reverse transferase-containing fraction obtained in the solubilization procedure is further purified.

Alternatively, the solubilization procedure can be carried out by sonicating insect cells obtained by the synthetic procedure, centrifuging the first sonicated product, removing the first supernatant, treating the resultant first precipitate with one or more detergents other than N-D-methylalkanamide of the formula (1), and then centrifuging the treated product, removing the second supernatant, and then using the resultant second precipitate as the starting material of the solubilization procedure. In this case, most of soluble proteins in the sonicated lysates can be removed by the first centrifugation step, and the residual soluble proteins remained in the precipitates of the first centrifugation step can be mostly fractionated to be soluble in the second centrifugation step. Therefore, it is possible to skip or simplify some separation steps to remove soluble proteins when soluble protein fractions (containing the telomerase reverse transcriptase) are purified from the precipitates of the second centrifugation step as mentioned above.

For example, the telomerase can be reconstituted in vitro by mixing the purified hTERT protein as mentioned above and independently prepared RT. Reference is made to, for example, Evaluation Example 1 (1) as mentioned below.

### EXAMPLES

The present invention will now be further illustrated by, but is by no means limited to, the following Examples.

### Example 1:

### Preparation of FLAG-hTERT Fusion Protein

(1)Construction of plasmid

A plasmid, pCI-Neo-hTERT, harboring hTERT cDNA (Nature, 400, 464-468, 1999) was cut with restriction enzymes EcoR1 and SalI, to generate an EcoR1/SalI DNA fragment harboring the hTERT-cDNA. By inserting the EcoR1/SalI DNA fragment into the EcoR1 and SalI sites of a plasmid, pNKFLAGZ, pNKFLAG-hTERT was constructed. The plasmid, pNKFLAGZ, was modified from a plasmid, pNKFLAG, (J. Biol. Chem., 273, 15479-15486, 1998) to generate the FLAG sequence followed by EcoR1 and SalI sites in frame.

The constructed plasmid, pNKFLAG-hTERT, harbored a DNA sequence encoding an hTERT fusion protein tagged by FLAG at N-terminal (FLAG-tagged hTERT).

By digestion of pNKFLAG-hTERT with NotI and Bg1II, a NotI/Bg1II DNA fragment including the DNA sequence encoding the FLAG-hTERT fusion protein was obtained. The NotI/Bg1II DNA fragment was inserted into the NotI and Bg1II sites of a baculovirus transfer vector, pVL1393 (Pharmingen), to generate a plasmid pBYK-FLAG-hTERT.

(2) Preparation of the recombinant baculoviruses

Preparation of the recombinant baculovirus was carried out, using a commercially available kit, BaculoGold Starter Package, including Sf9 insect cells and linealized BaculoGold baculovirus DNA (Pharmingen), as follows:

Sf9 cells cultured in suspension in TNM-FH insect cell culture medium supplemented with 10% fetal calf serum (hereinafter referred to as FCS), were inoculated at a cell density of 10⁶ cells/ 9 cm² dish 30 minutes before transfection. Then, co-transfection with the plasmid, pBYK-FLAG-hTERT, (2.5 μg), and the linealized BaculoGold baculovirus DNA (0.25 μg) was carried out according to the attached manual of the kit mentioned above. After the co-transfection, the cells were cultured for 5 days at 27° C., then the supernatant was recovered. Amplification of the recombinant virus, BVKM-FLAG-hTERT strain, was carried out with the recovered supernatant and Sf9 cells. Virus titer was measured with Sf9 cells according to the attached manual of the kit. The following experiments were carried out with high titer stocks of the BVKM-FLAG-hTERT baculovirus strain having 1.0×10⁷ pfu/mL or more. The stocks of the recombinant BVKM-FLAG-hTERT baculovirus strain were stable at 4° C. for about 6 months, but then the titers were gradually reduced.

(3) Evaluation of FLAG-hTERT expression levels in various insect cells

Expression level of the FLAG-hTERT fusion protein was evaluated with Sf9 used in Example 1 (2) and High5 cells (Invitrogen). The medium for the Sf9 cells was TNM-FH supplemented with 10% FCS as mentioned in Example 1 (2), and that for the High5 cells was prepared by adding 10% FCS to a commercially available medium (High Five serum free medium, Invitrogen). The recombinant baculovirus BVKM-FLAG-hTERT strain was infected to the cells at multiplicity of infection (moi) of 2, and thereafter the cells were cultured for 3 days at 27° C.

Expression of the FLAG-hTERT fusion protein was observed in both of the infected Sf9 and High5 cells by Western blotting with an anti-FLAG monoclonal antibody (SIGMA). These expression levels of the protein were much higher than those detected in bacterial or mammalian cells which were evaluated by the present inventors. When Coomassie brilliant blue staining (hereinafter referred to as CBB), which is a less sensitive detection method, was carried out, the FLAG-hTERT fusion protein expressed in High5 cells was detected, whereas the expression in the Sf9 cells was not detected. Much higher expression of the fusion protein was indicated in High5 cells than in Sf9 cells.

(4) Determination of optimal conditions of multiplicity of infection (moi) and incubation time after infection

Optimum conditions of multiplicity of infection (moi) and incubation time after infection were determined with High5 cells.

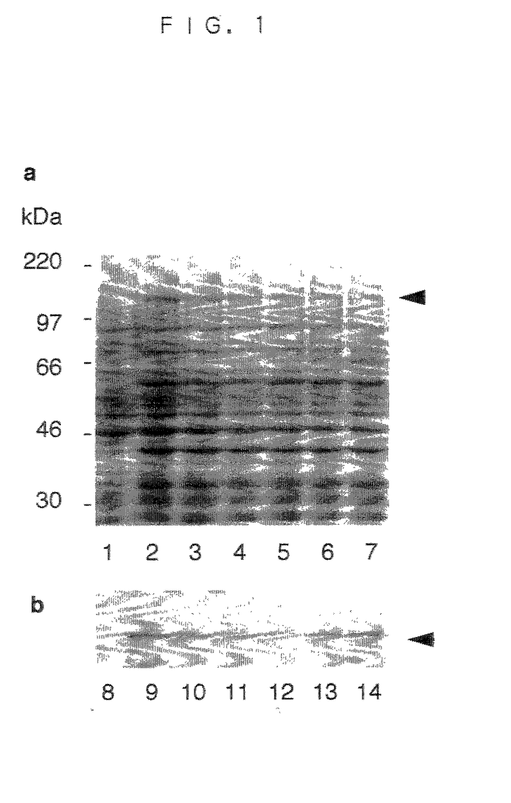

A result of the recombinant BVKM-FLAG-hTERT baculovirus infection at varying multiplicity of infection is shown in FIG. 1. FIG. 1a shows profiles obtained by recovering total cell lysates of the cell cultured for 5 days after infection, fractionating the proteins by Sodium Dodecyl Sulfate (hereinafter referred to as SDS)-polyacrylamide gel electrophoresis (hereinafter referred to as PAGE) (8% gel concentration), and then subjecting to CBB staining. FIG. 1b shows a result of Western blotting with the anti-FLAG-monoclonal antibody (SIGMA) for the fractionated products obtained as in FIG. 1a. The arrows in FIGS. 1a and 1b indicate FLAG-hTERT. Lanes 1, 8: Total lysates of non-infected insect cells. Lanes 2-7, and 9-14: Total lysates of the insect cells infected with BVKM-FLAG-hTERT at different moi; moi of lanes 2 and 9, 3 and 10, 4 and 11, 5 and 12, 6 and 13, and 7 and 14, is 0.2, 0.5, 1, 2, 5, and 10, respectively.

Usually, the optimum multiplicity of infection with recombinant baculovirus has been regarded to be 5 to 10. However, as shown in FIG. 1, the expression of the FLAG-hTERT fusion protein in the cells infected at moi of 0.2 is similar to or higher than that in the cells infected at moi of 5 to 10. Although data are not shown herein, when moi was reduced to 0.1 to 0.2, a yield of the FLAG-hTERT fusion protein was reduced, and therefore, the optimum moi seems to be approximately at 0.2.

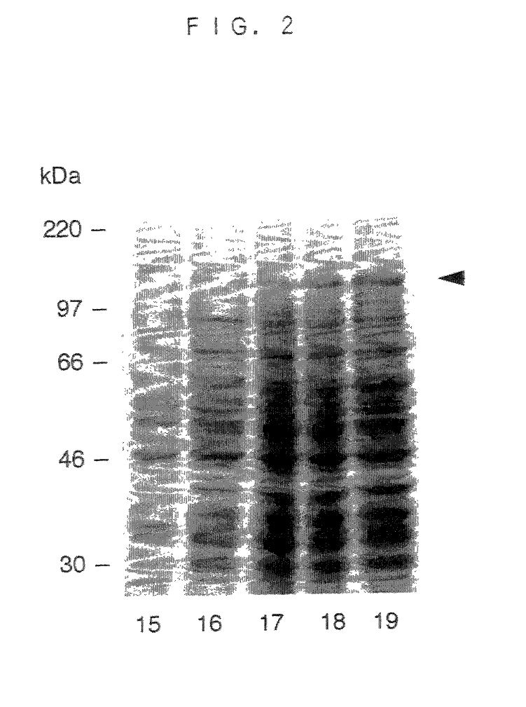

FIG. 2 shows the results obtained by culturing the insect cells infected with BVKM-FLAG-hTERT at moi of 0.2 for 48 hours, 72 hours, 96 hours, 120 hours, or 144 hours, fractionating total lysates of the insect cells with SDS-PAGE, and staining the fractionated product with CBB. Lanes 15 to 19 are the results of the cells harvested at time of 48 hours, 72 hours, 96 hours, 120 hours, and 144 hours of post infection, respectively. The positions of the FLAG-hTERT fusion proteins are shown by arrow.

As shown in FIG. 2, the maximum expression of the FLAG-hTERT fusion protein was detected at 4 to 6 days after infection.

(5) Expression and purification of the FLAG-hTERT fusion protein

For protein expression of FLAG-hTERT, 1.0×10⁷ High5 cells (Invitrogen) cultured in a commercially available medium (High Five serum-free medium; Invitrogen) seeded onto 5×25 cm² dishes before infection. The cells were infected with the recombinant baculovirus, BVKM-FLAG-hTERT, prepared in Example 1 (2) at moi of about 0.2. These infected High5 cells were incubated for 5 days at 27° C.

The infected cells were scraped off the plate, suspended with Phosphate buffered saline (hereinafter referred to as PBS(−)), and centrifuged at 4,000 rpm for 10 minutes.

All subsequent steps were performed at 4° C., and all buffers contained 1 mmol/L phenylmethylsulfonyl fluoride (SIGMA), 10 μg/mL pepstatin A (SIGMA), 10 μg/mL leupeptin (SIGMA), 10 μg/mL aprotinin (BOEHRINGER MANNHEIM), 10 μg/mL phenanthrorin (SIGMA), 16 μg/mL benzamide (SIGMA), and 1 mmol/L DTT (Nakalai Tesque). As a control, High5 cells without infected with BVKM-FLAG-hTERT were harvested and subjected to the same purification steps as those with the infected cells.

The collected cells were resuspended in 5 mL of buffer A (20 mmol/L Tris-HCl, pH 7.5, 20% glycerol, 0.1% Nonidet P-40, 150 mmol/L NaCl, 10 mmol/L β-mercaptoethanol) and the suspension was sonicated 3 times for 10 seconds. After a 10 minutes centrifugation at 10,000 g, the supernatant (hereinafter referred to as S1) was removed, the pellet was resuspended in 5 mL of the lysis buffer A once again.

After a centrifugation at 10,000 g for 10 minutes, the supernatant was discarded, the pellet was resuspended in 1 mL of lysis buffer C (20 mmol/L Tris-HCl, pH 7.5, 50% glycerol, 0.5% MEGA-9, 500 mmol/L NaCl, 10 mmol/L β-mercaptoethanol) and the suspension was sonicated three times for 10 seconds. After a centrifugation at 10,000 g for 10 minutes, supernatant (hereinafter referred to as S2) was removed and the pellet was resuspended in 1 mL of lysis buffer 4 C (20 mmol/L Tris-HCl, pH 7.5, 50% glycerol, 0.5% MEGA-9, 1000 mmol/L NaCl, 10 mmol/L β-mercaptoethanol).

The suspension was sonicated three times for 10 -seconds and after a centrifugation at 10,000 g for 10 minutes, supernatant (hereinafter referred to as S3) was collected. The supernatants S2 and S3 were mixed together. The mixture was diluted with buffer D [20 mmol/L Tris-HCl, pH 7.5, 50% glycerol, 0.5% MEGA-9, 1 mmol/L DTT (dithiothreitol)] to adjust an NaCl concentration to 100 mmol/L (hereinafter referred to as S4).

The fraction S4 was passed through a DEAE-Sepharose equilibrated with buffer E (20 mmol/L Tris-HCl, pH 7.5, 50% glycerol, 0.5% MEGA-9, 100 mmol/L NaCl, 1 mmol/L DTT). After equilibrated with buffer E, a mixture (1 mL) of Heparin-Sepharose CL-6B and Sepharose CL-6B at a mass ratio of 1:1 (hereinafter referred to as Heparin-Sepharose) was added to the flow-through fractions. The mixture was rotated for 3-4 hours at 4° C.

Proteins bound to Heparin-Sepharose were washed with the buffer E and then eluted with 2 mL of the buffer B to obtain the purified FLAG-hTERT fusion protein. For quantification, the purified FLAG-tagged hTERT protein was separated by SDS-PAGE (8% polyacrylamide gel) and gels were stained with CBB. Bovine serum albumin (SIGMA) was used as a standard protein to estimate concentrations of the sample protein.

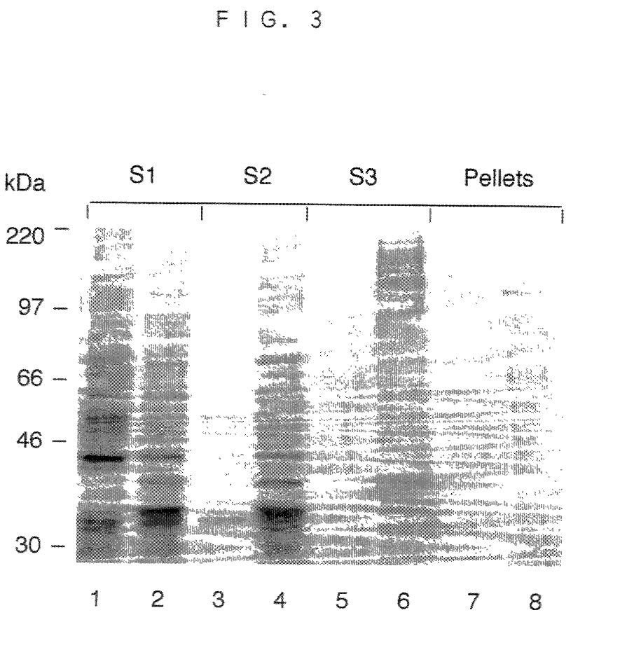

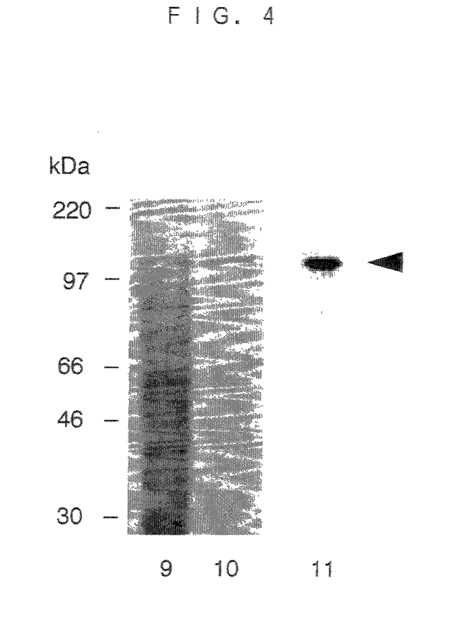

The fractionated samples were separated by SDS-PAGE (8% polyacrylamide gel) and subjected to CBB staining as shown in FIG. 3. The final purified sample of the FLAG-hTERT fusion protein detected by CBB staining and Western blotting with anti-FLAG monoclonal antibody is shown in FIG. 4.

“S1”, “S2”, and “S3” in FIG. 3 denote the fraction S1, the fraction S2 and the fraction S3, respectively, and “Pellets” indicates the precipitates remaining after the fraction S3 was obtained. Lanes 1, 3, 5, and 7: Fractions of non-infected insect cells. Lanes 2, 4, 6, and 8: Fractions of FLAG-hTERT-expressing insect cells.

In FIG. 4, the total lysates of the infected cells (lane 9) and the Heparin Sepharose fraction (lane 10) were fractionated as in FIG. 3. The partially purified FLAG-hTERT (the same sample of lane 10 of FIG. 3) was subjected to Western blotting with anti-FLAG M2 antibody (lane 11).

As apparent from FIG. 3, most proteins of insect cells were eluted with the buffer A containing low concentrations of NP-40, and NaCl in glycerol (S1 and wash of the precipitates in the buffer A). In contrast, the FLAG-hTERT protein was only efficiently solubilized with the lysis buffer B containing MEGA-9 and a high concentration of salt (S3 and S4).

### Evaluation Example 1:

### In Vitro Reconstitution of Telomerase and Measurement of Telomerase Activity

(1) Reconstitution of telomerase in vitro

The present Evaluation Example was designed to directly prove that the recombinant FLAG-hTERT fusion protein prepared in the Example 1 retains the catalytic activity of telomerase by evaluating whether the mentioned FLAG-hTERT fusion protein and purified human telomerase RNA (hTR) can reconstitute the telomerase in vitro to exhibit a telomerase activity. Two different methods were applied to measure the telomerase activity; Namely, a telomere repeat amplification protocol assay (hereinafter referred to as TRAP assay) using a commercially available kit (TeloChaser; Toyobo), and a TRAP ELISA method, an enzyme liked immunosorbent assay of telomerase activity, using a commercially available kit, TRAPEZE kit (Intergen). Among these mentioned methods, a relative telomerase activity can be quantitated by the latter one, the TRAP ELISA method.

At first, human telomerase RNA, hTR, was synthesized by T7 RNA polymerase with an in vitro transcription system from a plasmid, pGRN164, harboring hTR-cDNA as a template.

In vitro reconstitution was carried out by mixing serially diluted recombinant FLAG-hTERT prepared in Example 1 and the in vitro transcribed hTR in 20 μL of reconstitution buffer A (final concentrations: 10 mmol/L HEPES-HCl, pH 8.0, 100 mmol/L NaCl, 25% glycerol, 1 mmol/L MgCl₂, 3 mmol/L KCl, 0.1 mmol/L PMSF, 1 mmol/L DTT, 10 U/mL RNasin) and incubating the whole at 33° C. for 10 minutes.

(2) Measurement of telomerase activity using TRAP assay

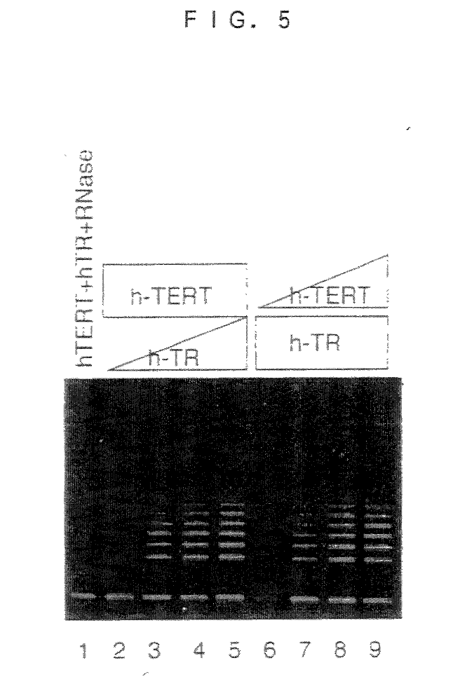

The results of the telomerase activity detected by the TRAP assay with the commercially available kit (TeloChaser; Toyobo) are shown in FIG. 5. The TRAP assay was carried out according to the manufacturer's manual. The PCR products were fractionated by electrophoresis on a 10% polyacrylamide gel, and then stained with SYBR Green I (Molecular Probes) to be visualized.

In FIG. 5, Lane 1 shows the result of the TRAP assay after preincubating 200 ng of hTR and 240 ng of FLAG-hTERT with 1 μl of RNase A (1 mg/mL) for 15 minutes at 30° C. Lanes 2 to 5 show the results of the TRAP assays after performing telomerase reactions of 120 ng of the partially purified FLAG-hTERT and 0 ng, 25 ng, 100 ng or 200 ng of the in vitro transcribed hTR, respectively, and Lanes 6 to 9 show the results of the TRAP assays after performing telomerase reactions of 200 ng of the in vitro transcribed hTR, and 0 ng, 30 ng, 120 ng or 240 ng of FLAG-hTERT, respectively.

As shown clearly in FIG. 5, the telomerase activity was detected by the TRAP assay only when FLAG-hTERT and hTR were present, whereas the presence of FLAG-hTERT alone exhibits no activity. This result indicates that the in vitro reconstitution of two components exhibits the telomerase activity. Further, this result clearly demonstrates that the telomerase activity can be observed by reconstitution of the two components, hTERT and hTR, in vitro. It also indicates that the two components are essentially minimum factors required for the telomerase activity.

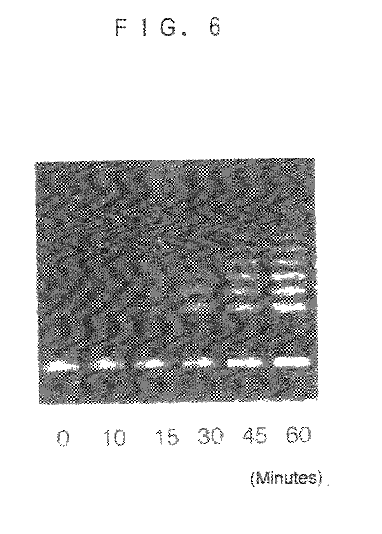

Time course of the telomerase reaction in the TRAP assay was examined as shown in FIG. 6. The telomerase reaction of FLAG-hTERT (120 ng) and hTR (200 ng) was carried out by incubating at 33° C. After the incubation as indicated in FIG. 6, aliquots were taken and subjected to PCR. The TRAP assay was carried out under the conditions as mentioned in Evaluation Example 1 (1) except for the reaction time.

As shown clearly in FIG. 6, the telomere synthesis was observed at 10 minute after the reaction start, then continued at least for 1 hour in an apparently linearly increasing fashion at 33° C. Since the same lag time was observed when the two components were preincubated at 33° C. without the template and the primer, the results suggest that one or more important conformation changes of the FLAG-hTERT fusion protein occur after the addition of the template and the primer.

The FLAG-hTERT exhibits a maximum catalytic activity at 30 to 37° C. at pH of around 8.0, while requiring magnesium ion at 2 mmol/L. The same optimum conditions for the telomerase activity were observed in the FLAG-hTERT fusion protein complemented by TIG3 cell extracts (data not shown).

(3) Measurement of telomerase activity by TRAP ELISA

As the TRAP assay carried out in Evaluation Example 1 (2) is sensitive, but not suitable to measure quantitatively the telomerase activity. In this Evaluation Example, the telomerase activity in the presence of the recombinant FLAG-hTERT protein and hTR at various molar ratios was measured by a TRAP ELISA assay using a commercially available kit, TRAPEZE kit (Intergen). The following procedures were carried out as directed by the manufacturer's instructions.

The components for the in vitro reconstitution were incubated in 20 μl of a reconstitution buffer B (final concentrations: 10 mmol/L HEPES, pH 8.0, 100 mmol/L NaCl, 25% glycerol, 1 mmol/L MgCl₂, 3 mmol/L KCl, 0.1 mmol/L PMSF, 1 mmol/L DTT, 10 U/μl RNasin, 2 ng/μl TS primer, 1 mmol/L non-labeled dATP, 1 mmol/L non-labeled dTTP, 0.1 mmol/L non-labeled dGTP, and 0.1 μCi/nL [α-³²P] labeled dGTP (800 Ci/mmole; Amersham Pharmacia Biotech) at 33° C. for one hour and 10 μl of the reaction was spotted onto a DE81 filter. The filter was washed and a level of a radioactivity bound to the filter was measured according to the methods reported previously.

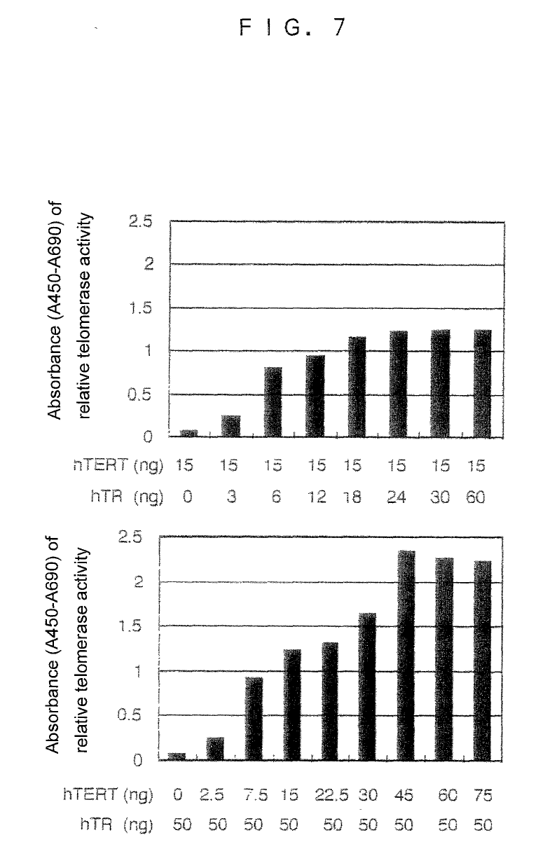

As shown in FIG. 7, telomerase exhibited a maximum activity when the two components, FLAG-hTERT and hTR, were present in an approximately equimolar ratio in the reaction. This result suggests an efficient complex formation of the two components in vitro.

### Evaluation Example 2:

### Complementation Assay of Telomerase activity Using the Telomerase-negative TIG3 Extracts and the FLAG-hTERT Fusion Protein

TERT has been reported to be a rate-limiting factor in tissue-cultured cells. In the present Evaluation Example, whether or not telomerase-negative TIG3 cell extract was complemented by the telomerase-active FLAG-hTERT fusion protein as to the telomerase activity was evaluated.

As a negative control, a mutant FLAG-hTERT fusion protein [hereinafter referred to as mutant FLAG-hTERT (D712)] was prepared. In the mutant FLAG-hTERT (D712), a VDD sequence, highly conserved among reverse transcriptases, was substituted to VAV, namely aspartic acid residue (D) at the 712nd amino acid in hTERT amino acid sequence was substituted to alanine residue (A).

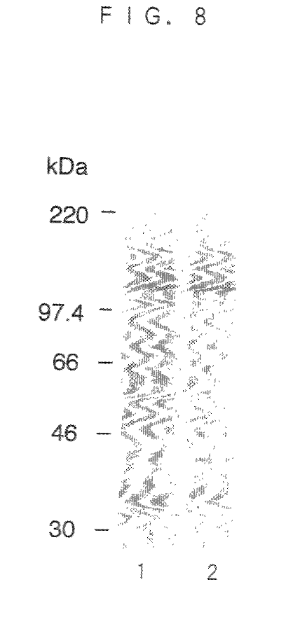

The mutant FLAG-hTERT (D712A) fusion protein was prepared by repeating the procedures mentioned in Example 1 (2) and Example 1 (5) except a plasmid harboring cDNA encoding the mutant FLAG-hTERT (D712A) was used. The FLAG-hTERT fusion protein prepared in Example 1 and the resulting mutant FLAG-hTERT (D712A) fusion protein were applied by SDS-PAGE (8% gel concentration), and subjected to CBB staining as shown in FIG. 8. In FIG. 8, lane 1 represents the FLAG-hTERT fusion protein, and lane 2 the mutant FLAG-hTERT (D712A) fusion protein, respectively.

Separately, TIG3 cells (HSRRB) were cultured in DMEM medium supplemented with 10% FCS and harvested. The recovered cells were suspended in a solubilization buffer [10 mmol/L HEPES (pH 8.00, 100 mmol/L NaCl, 0.5% MEGA-9, 25% glycerol, 1 mmol/L MgCl₂, 3 mmol/L KCl, 0.1 mmol/L PMSF, 1 mmol/L DTT, 10 U/μl RNasein] to obtain a solubilized product, i.e., TIG3 cell lysates.

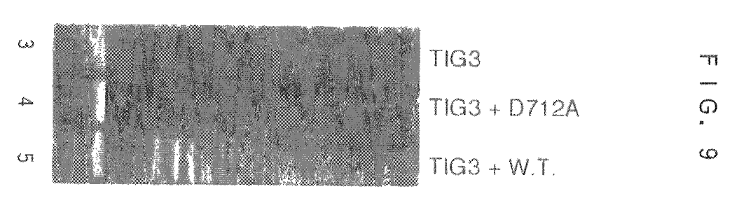

Aliquots of 90 ng of wild-type FLAG-hTERT (lane 5 in FIG. 9) or FLAG-hTERT(D712A) (lane 4 in FIG. 9) were incubated with TIG3 extracts (corresponding to 1.0×10⁴ of TIG3 cells) (total volume of reaction was 20 μl), and telomerase reactions were carried out at 33° C. for 1 hour, and then the reaction products were subjected to PCR reaction.

The result is shown in FIG. 9. Lane 3 of FIG. 9 is the result of cell extracts from 1.0×10⁴ of TIG3 cells alone. Lane 4 is the result of the telomerase reaction of the TIG3 extract and the mutant FLAG-hTERT (D712A), and lane 5 is the result of the telomerase reaction of the TIG3 extract and the wild-type FLAG-hTERT.

As clearly indicated in FIG. 9, the telomerase activity was not detected in the case wherein the TIG3 cell extract was used alone, but was detected only when the wild FLAG-hTERT fusion protein was added to the TIG3 cell extract. Addition of the mutant FLAG-hTERT (D712A) fusion protein to the TIG3 extract did not complement the telomerase activity. These results indicate that the purified FLAG-hTERT protein retains the catalytic activity of human telomerase, and complements the telomerase activity of telomerase-negative cells. The mutant FLAG-hTERT (D712A) protein did not exhibit the telomerase activity in the in vitro reconstitution experiment (data not shown).

### Evaluation Example 3:

### Stimulatory Effect of TIG3 Cell Extracts on Telomerase Activity Reconstituted in Vitro

Serially diluted TIG3 cell extracts were added to the telomerase reconstituted from the FLAG-hTERT fusion protein (5 ng) and hTR (9 ng) as mentioned in Evaluation Example 1 (1), and then telomerase activity was measured by the TRAP assay as mentioned in Evaluation Example 1 (2) and TRAP ELIZA assay as mentioned in Evaluation Example 1 (3).

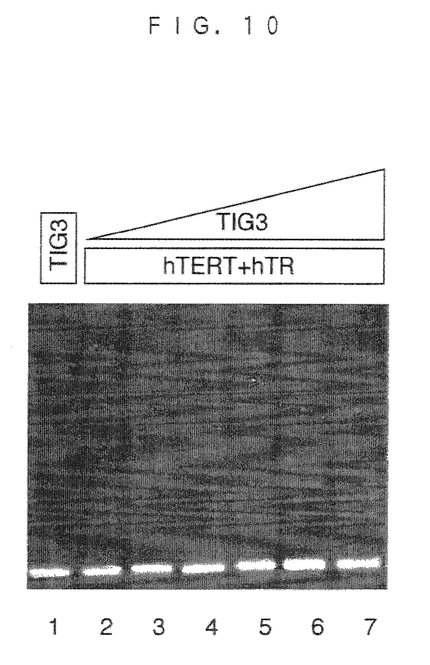

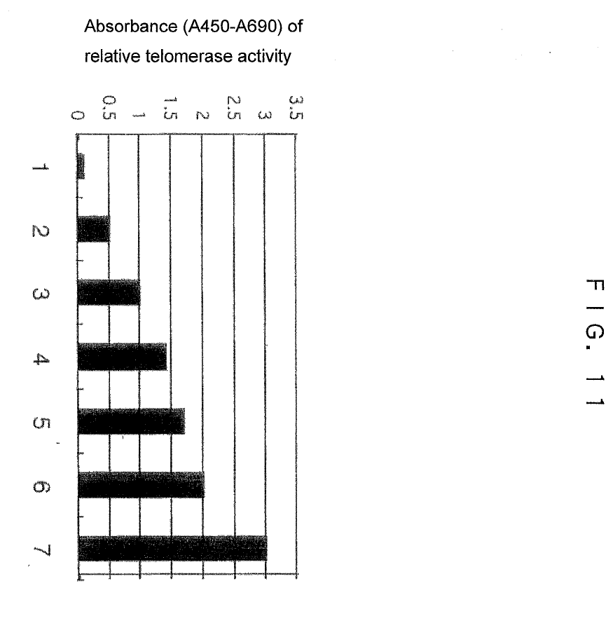

The results of the telomerase activities detected by the TRAP and TRAP ELISA assays are shown in FIGS. 10 and 11, respectively. In each of FIGS. 10 and 11, lane 1 is the telomerase activity obtained in the case wherein the TIG3 cell extract (corresponding to 1.0×10⁴ TIG3 cells) was used alone, lanes 2 to 7 represent the telomerase activities obtained in the cases wherein the TIG3 cell extracts were added to the in vitro reconstituted telomerase at an amount of 5.0×10² (line 2), 1.0×10³ (line 3), 2.0×10³ (line 4), 4.0×10³ (line 5), 8.0×10³ (line 6), and 1.6×10⁴ (line 7), respectively. As clearly shown in FIGS. 10 and 11, TIG3 cell extract stimulated the telomerase activity reconstituted in vitro, as detected by the two methods. The telomerase activity reconstituted by the FLAG-hTERT fusion protein (5 ng) and hTR (9 ng) was augmented by 30 fold or more in the presence of the TIG3 cell extracts in a dose dependent manner. This result strongly suggests that the TIG3 cells contain some factors which stimulate the telomerase activity reconstituted from the FLAG-hTERT fusion protein and hTR in vitro.

### Evaluation Example 4:

### Effect of an Inhibitor Against Hsp90 on Telomerase Activity in Vitro

Recently, Hsp90 and p23 have been reported to be essential for telomerase activity, relying on the experiments using recombinant hTERT de novo synthesized in a rabbit reticulocyte system. In the present Evaluation Example, an effect of Geldanamycin (hereinafter referred to as GA), investigations were made in a specific inhibitor against Hsp90, on telomerase activity reconstituted in vitro and stimulated in the presence of the TIG3 cell extract.

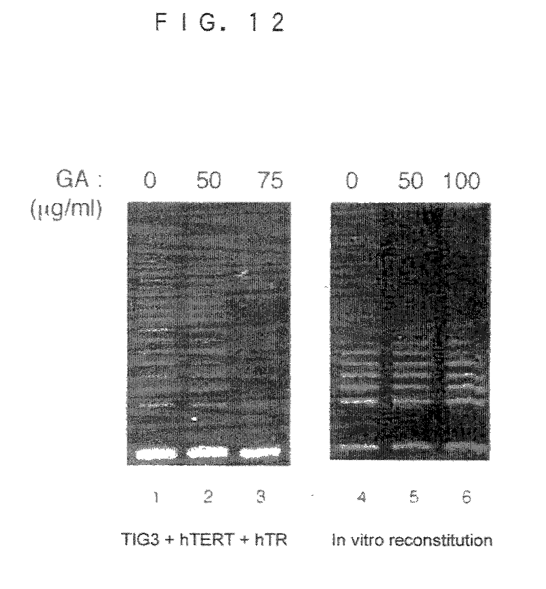

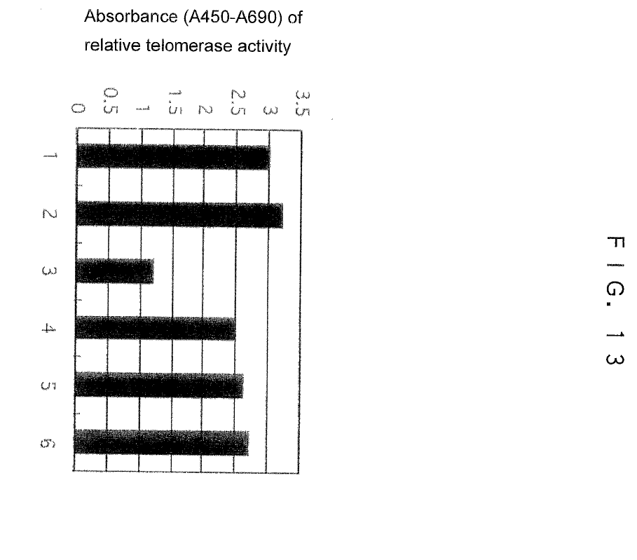

The telomerase activity examined by the TRAP assay (reaction time=1 hour, reaction temperature=33° C.) is shown in FIG. 12, and that detected by the TRAP ELISA is shown in FIG. 13, respectively. In each of FIGS. 12 and 13, lanes 1 to 3 represent the telomerase activities in the cases wherein the TIG3 extracts (10⁴ cells) and GA at various concentrations were added to the FLAG-hTERT (5 ng) and hTR (9 ng) reconstituted in vitro. Lanes 4 to 6 represent the telomerase activities in the cases wherein GA at various concentrations was added to the FLAG-hTERT (5 ng) and hTR (9 ng) reconstituted in vitro.

As clearly shown in FIGS. 12 and 13, GA at higher concentrations was able to slightly inhibit the telomerase activities stimulated by TIG3 cell extracts (lanes 1 to 3). In contrast, the telomerase activity was not inhibited at all by Geldanamycin even in higher concentrations (up to 100 μg/mL) when it was added before (lanes 4-6) or after (data not shown) reconstitution of FLAG-hTERT and hTR in vitro. In consistent to the latter result, Hsp90 was not detected in the purified preparation of FLAG-hTERT by CBB staining (FIGS. 2 and 8) or anti-Hsp90 antibody (data not shown). These results strongly suggest that the TIG3 extract probably contains, in addition to Hsp 90 (and probably p23), some factors to stimulate telomerase activity reconstituted in vitro.

### Evaluation Example 5:

### Complex Formation of the FLAG-hTERT Protein and hTR in Vitro

In this Evaluation Example, a complex formation of the FLAG-hTERT protein and hTR in vitro was directly detected by a gel shift assay (Electrophoretic Motility Shift Assay: hereinafter referred to as EMSA).

Human TR (consisting of 483 nucleotides) and a control RNA, SG-RNA (560 nucleotides) were prepared with T7 in vitro transcription system as mentioned previously using hTR-cDNA (pGRN164) and pSG5UTPL-p53 (Cancer Res. 57, 5137-5142, 1997) as templates, respectively. In detail, linearized plasmids, pGRN164 and pSG5UTPL-p53²⁰, with FspI and with BalI respectively, were transcribed by T7 RNA polymerase using RiboMAX RNA Production System (Promega) according to the manufacturer's manual attached to the kit. For labeling the probes, 1 μL of [(α-³²p] UTP (10 mCi/ml, 800 Ci/mmol; Amersham Pharmacia Biotech) was added to the reaction as a substrate.

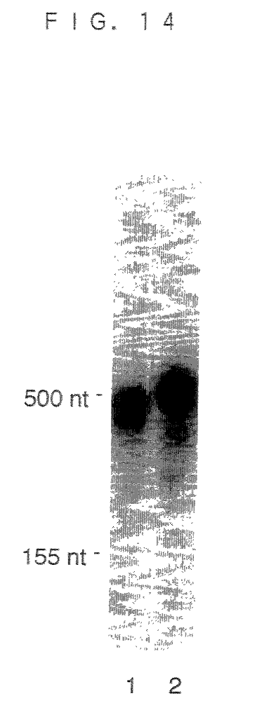

The synthesized ³²P-labeled hTR and the control RNA were fractionated by 4% polyacrylamide gel electrophoresis in the presence of 7 mol/L Urea as shown in FIG. 14. Lane 1 of FIG. 14 is the fractionated hTR RNA, and lane 2 is the fractionated control RNA.

Binding reactions were done at 33° C. for 1 hour by incubating various amount of hTERT and 2 ng of labeled RNA in 20 μL of a binding buffer (10 mmol/L HEPES, pH 8.0, 100 mmol/L NaCl, 0.5% NP-40, 25% glycerol, 4 mmol/L MgCl₂, 3 mmol/L KCl, 0.1 mmol/L PMSF, 1 mmol/L DTT, 10 U/μL RNasin) together with a non-labeled RNA (at various amounts), a competitive component.

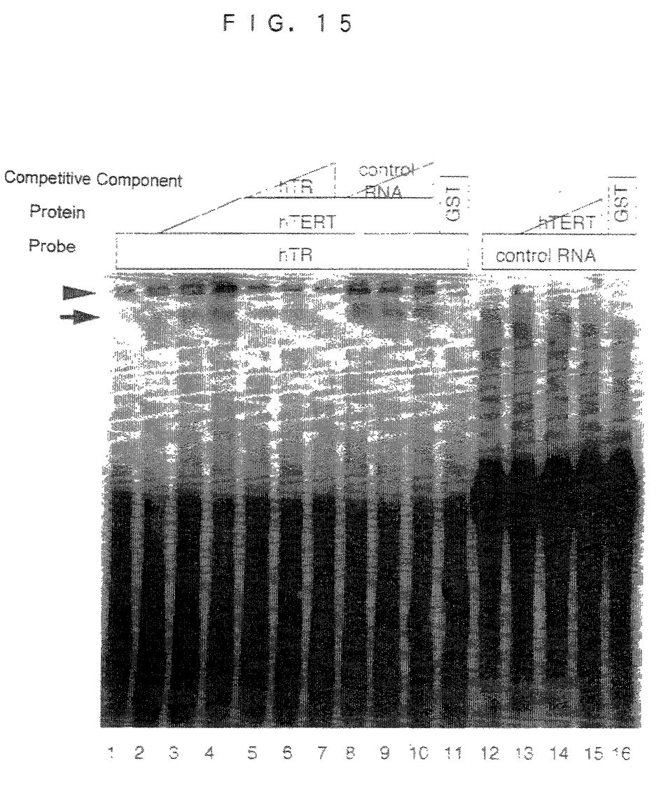

The reaction products separated by native gel electrophoresis are shown in FIG. 15. In FIG. 15, “hTERT” represents the FLAG-hTERT fusion protein, “control RNA” represents the control RNA, namely SG-RNA, and “GST” represents glutatione-S-transferase (hereinafter referred to as GST), respectively. The arrow indicates the position of the complex of the FLAG-hTERT fusion protein and the labeled hTR probe. The solid triangle denotes the position of the probe not contained in the gel.

In FIG. 15, lane 1 is the result of the case of only the labeled hTR probe (2 ng). Lanes 2-4 are the results of the cases wherein a constant amount of the labeled hTR probe (2 ng) was mixed with the FLAG-hTERT fusion protein at an amount of 1 ng (lane 2), 5 ng (lane 3), and 10 ng (lane 4), respectively. Lanes 5-7 are the results of the cases wherein a constant amount of the labeled hTR probe (2 ng) and a constant amount of the FLAG-hTERT fusion protein (10 ng) were mixed with the non-labeled hTR at an amount of 4 ng (lane 5), 10 ng (lane 6) and 20 ng (lane 7), respectively. Lanes 8-10 are the results of the cases wherein a constant amount of the labeled hTR probe (2 ng) and a constant amount of the FLAG-hTERT fusion protein (10 ng) were mixed with the non-labeled control RNA at an amount of 4 ng (lane 8), 10 ng (lane 9) and 20 ng (lane 10), respectively. Lane 11 is the result of the case wherein the labeled hTR (2 ng) was mixed with GST (20 ng) and lane 12 is the result of the case of only the labeled control RNA (2 ng). Lanes 13-15 are the results of the cases wherein a constant amount of the labeled control RNA probe (2 ng) was mixed with the FLAG-hTERT fusion protein at an amount of 1 ng (lane 13), 3 ng (lane 14), and 6 ng (lane 15), respectively. Lane 16 is the result of the case wherein a constant amount of the labeled control RNA probe (2 ng) was mixed with GST (20 ng).

The hTR probe became to be a single band in denaturating gels (FIG. 14, lane 1) but it made multimeric forms in the absence of protein in native gels probably due to intermolecular interaction of hTR (FIG. 15, lane 1). As shown in FIG. 15, these free bands of the hTR were diminished by FLAG-hTERT in a dose dependent manner, but the specific complex formation of the labeled hTR probe and the FLAG-hTERT was observed, and the amount of the portion not entered into the gel was increased dose-dependently. These complexes were eliminated by excess amount of non-labeled hTR, but not by the control SG-RNA. No specific complex formation was observed in the combinations of the control SG-RNA and the FLAG-hTERT fusion protein, or of the hTR and GST. These results strongly suggest that hTERT and hTR can form the specific complex and the complex probably consists of equimolar amount of the two components since a free hTR probe was not substantially observed when equimolar amounts of FLAG-hTERT and hTR were present.

### INDUSTRIAL APPLICABILITY

According to the synthetic procedure by the present invention, it is possible to prepare a large amount of TERT. For example, it is possible to recover approximately 2 mg of hTERT from 10⁸ infected cells if the optimum multiplicity of infection and incubation time after infection are applied.

Also, insoluble hTERT synthesized in the synthetic procedure mentioned above can be solubilized by the solubilization procedure of the present invention.

According to the production method of the present invention, a large amount of hTERT that enables to prepare a large amount of reconstituted telomerase in the presence of hTR can be produced.

The present invention including the mentioned procedures is expected to be applied to developing drug designs molecularly targeting telomerase to prevent cell scenscense and oncogenesis. Also, human telomerase reconstituted in vitro can contribute to identify novel factors in telomerase-negative cells which may be involved in activity and/or regulation of telomerase.

As above, the present invention is explained with reference to particular embodiments, but modifications and improvements obvious to those skilled in the art are included in the scope of the present invention.

What we claim is:

1. A method for producing telomerase reverse transcriptase comprising the steps of:
infecting an insect cell with a baculovirus containing a DNA encoding telomerase reverse transcriptase, and then culturing the infected cell to express the telomerase reverse transcriptase.

2. A method for solubilizing telomerase reverse transcriptase comprising treating an insect cell expressing telomerase reverse transcriptase or a lysate thereof with an aqueous solution containing N-D-gluco-N-methylalkanamide of the formula (I): 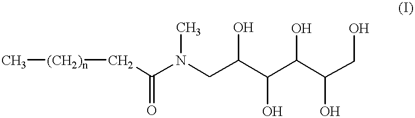
wherein n denotes an integer of 5 to 7.

3. A method for producing telomerase reverse transcriptase comprising the steps of:
infecting an insect cell with a baculovirus containing a DNA encoding telomerase reverse transcriptase,
culturing the infected cell to express the telomerase reverse transcriptase, and then,
treating the insect cell expressing telomerase reverse transcriptase or a lysate thereof with an aqueous solution containing N-D-gluco-N-methylalkanamide of the formula (I): 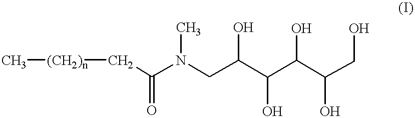
wherein n denotes an integer of 5 to 7.

4. A baculovirus expression vector containing a DNA encoding telomerase reverse transcriptase.

5. A baculovirus containing a DNA encoding telomerase reverse transcriptase.

6. An insect cell containing the baculovirus according to claim 5.
